(19) United States
(12) Patent Application Publication
(10) Pub. No.: US 2026/0029316 A1
(43) Pub. Date: Jan. 29, 2026

(54) METHOD FOR PRETREATMENT OF SPECIMEN FOR IMMUNOCHROMATOGRAPHIC TEST

(71) Applicant: DRUG DISCOVERY INSTITUTE FOR INFECTIOUS DISEASES CO., LTD., Tokyo (JP)

(72) Inventors: Koichi SHIRAISHI, Chuo-ku (JP); Sohei TANABE, Chuo-ku (JP); Masahiro FUSE, Chuo-ku (JP); Tadaaki OHGIYA, Chuo-ku (JP)

(73) Assignee: DRUG DISCOVERY INSTITUTE FOR INFECTIOUS DISEASES CO., LTD., Tokyo (JP)

(21) Appl. No.: 18/859,412

(22) PCT Filed: May 31, 2023
(86) PCT No.: PCT/JP2023/020235

§ 371 Date: Oct. 23, 2024

(30) Foreign Application Priority Data
May 31, 2022 (JP) 2022-088484

(51) Int. Cl. G01N 1/40; G01N 33/543; G01N 33/569

(52) U.S. Cl. CPC G01N 1/4077; G01N 1/4005; G01N 33/54306; G01N 33/56983; G01N 2001/4016; G01N 2001/4088; G01N 2333/165; G01N 2333/4725

(57) ABSTRACT

An immunochromatographic method for a trace amount of an antigen or an antibody such as SARS-COV-2 including use of saliva, enabling highly sensitive immunochromatographic antigen or antibody detection even when the saliva is used. A method for pretreatment of a specimen for an immunochromatographic test, including passing the saliva through a porous member capable of supporting a viscous component in the saliva to remove the viscous component, passing the liquid that has passed through the porous member through a filter having a hole diameter of from 0.1 μm to 10 μm, and concentrating an antigen or an antibody in the liquid that has passed through the filter.

### Field of the Invention

The present invention relates to a method for pretreatment of a specimen for an immunochromatographic test of saliva and a kit for the pretreatment, and an immunochromatographic test method.

### BACKGROUND OF THE INVENTION

COVID-19, which is a novel coronavirus infectious disease, is an infectious disease whose onset was first observed near Wuhan, China in 2019 and which caused a worldwide pandemic thereafter. At present, an IgG antibody, an IgM antibody, and a neutralizing antibody, which are each a kind of immunoglobulin in blood that specifically reacts with the nucleocapsid protein and spike protein (hereinafter referred to as “S antigen”) of SARS-COV-2, have already been observed, and an antiviral drug, a neutralizing antibody drug, an immunomodulator, or the like has been developed as a pharmacotherapy for the COVID-19. Meanwhile, a vaccine for preventing the onset of the COVID-19 has been developed, and the effect of the inoculation of the vaccine has been recognized. However, a noninvasive method facilitating the recognition of an antibody fluctuation before and after the inoculation of the vaccine, and the judgment of the timing of the next inoculation of the vaccine is absent, and hence its practical application has been required.

With regard to an antibody to the SARS-COV-2, measuring equipment adopting chemiluminescent immunoassay (CLIA), which is an immunoassay, as a principle, and a qualitative test kit for the antibody in whole blood, serum, or plasma on the basis of an immunochromatographic method as a test instrument that judges the presence or absence of the antibody have been developed, and a test for evaluating the performance of the kit has been performed (Non Patent Document 1). The immunochromatographic method is excellent in simpleness and rapidity, and it can judge a diagnostic result in from about 10 to 20 minutes merely by dropping a specimen. Collection of a blood specimen is regarded as being relatively easy, but it is problematic as it is invasive to a subject. Accordingly, establishment of a method of the qualitative test for an antibody including use of a noninvasively collected biological specimen, e.g., saliva or nasal discharge, has been desired. In addition, collection of saliva or nasal discharge can be performed where availability of a collecting instrument is limited, or where strict hygiene practices may not be obeyed.

A problem in a general immunoassay including use of a high-viscosity biological specimen, e.g., saliva or nasal discharge that can be noninvasively collected, is that an accurate result may not be obtained owing to a nonspecific reaction or a wrong result e.g., a false positive is obtained. Some saliva-collecting instruments that are currently available in the market can remove mucus in the saliva (Patent Document 1), but are each deficient in simpleness because the instruments each require centrifugation treatment with a centrifuge. In addition, some instruments remove viscous components through use of their filter functions (Patent Document 2), but sufficient detection sensitivity is not obtained when a measuring object is, for example, an antibody that is present only in an extremely small amount in the saliva.

With regard to a method of solving the nonspecific reaction, a method including use of a pretreatment liquid containing a nonionic surfactant and an alkali metal ion (Patent Document 3), and a method including use of a pretreatment liquid containing a water-soluble ammonium polymer or the like (Patent Document 4) are disclosed. However, these methods need pre-confirmation whether they influence on a test, it is assumed that the pretreatment liquid may dilute a target antibody titer (the amount of an antibody with respect to an antigen).

There is a correlation between the neutralizing action of a SARS-COV-2 antibody on the S antigen in saliva and that in serum (Non Patent Document 2). However, it was reported that the titer of the antibody in the saliva is lower than that in blood by a factor of 1 000 or more (Non Patent Document 3). Accordingly, the titer may not be accurately measured by a conventional immunochromatographic method which employs saliva as a specimen.

### PRIOR ART DOCUMENTS

### Patent Documents

[Patent Document 1] U.S. Pat. No. 4,774,962 A

[Patent Document 2] JP-B2-4131966

[Patent Document 3] JP-A-2005-24323

[Patent Document 4] JP-B2-5334742

### Non Patent Documents

[Non Patent Document 1] Report on simultaneous test for evaluating performances of antibody test kits for novel coronavirus (Jul. 14, 2021, National Institute of Health Sciences)

[Non Patent Document 2] Microbiol Spectr. 2021 Oct. 31; 9(2): e0069321

[Non Patent Document 3] Pathog Immun. 2021 Jun. 7; 6(1): 116-134

### SUMMARY OF THE INVENTION

Accordingly, the present invention aims to provide an immunochromatographic method for a trace amount of an antigen or an antibody such as SARS-COV-2 including use of saliva, the method enabling highly sensitive immunochromatographic antigen or antibody detection even when the saliva is used.

In view of the foregoing, the present inventors made various investigations with a view to simply and efficiently fractionating a high concentration of a target antigen or a target antibody from saliva. As a result, the present inventors found that when the saliva was subjected to the following three steps (1) to (3) in combination: (1) passing the saliva through a porous member capable of supporting the viscous component in the saliva to remove the viscous component; (2) passing the liquid that had passed through the porous member through a filter having a hole diameter of from 0.1 μm to 10 μm; and (3) concentrating an antigen or an antibody in the liquid that had passed through the filter, only a viscous component was to be removed in the step (1), the target antigen or the target antibody was able to be quickly filtered in the step (2), and the concentration of the target antigen or the target antibody in a specimen was sufficiently increased by the step (3), and hence the sensitivity of detection by an immunochromatographic method was dramatically improved. Thus, the present inventors completed the present invention.

That is, the present invention provides the following inventions [1] to [16].

[1] A method for pretreatment of a specimen for an immunochromatographic test, the method comprising subjecting saliva to:
(1) passing the saliva through a porous member capable of supporting a viscous component in the saliva to remove the viscous component;
(2) passing the liquid that has passed through the porous member through a filter having a hole diameter of from 0.1 μm to 10 μm; and
(3) concentrating an antigen or an antibody in the liquid that has passed through the filter.

[2] The method for pretreatment according to [1], wherein the porous member has a void configured to pass a low-viscosity component therethrough but not to pass a mucin in the saliva therethrough when a pressing force of 1 000 kPa or less is applied thereto.

[3] The method for pretreatment according to [1] or [2], wherein the porous member has a pore having a hole diameter of from 10 μm to 3 000 μm.

[4] The method for pretreatment according to any one of [1] to [3], wherein the porous member is disposed at a tip portion of a saliva-collecting tool.

[5] The method for pretreatment according to any one of [1] to [4], wherein the filter has 1 to 4 layers.

[6] The method for pretreatment according to any one of [1] to [5], wherein the concentrating the antigen or the antibody adopts affinity chromatography of the antigen or the antibody.

[7] The method for pretreatment according to any one of [1] to [6], wherein a target antigen or a target antibody is SARS-COV-2 or a SARS-COV-2 antibody.

[8] A method for an immunochromatographic test of an antigen or an antibody in saliva, the method comprising subjecting a specimen obtained by the method for pretreatment step according to any one of [1] to [7] to an immunochromatographic antigen or antibody test kit.

[9] A kit for pretreatment of a specimen for an immunochromatographic test, the kit comprising:
(a) a porous member capable of supporting a viscous component in saliva;
(b) a filter having a hole diameter of from 0.1 μm to 10 μm; and
(c) a device configured to concentrate an antigen or an antibody.

[10] The kit for pretreatment according to [9], wherein the porous member has a void configured to pass a low-viscosity component therethrough but not to pass a mucin in the saliva therethrough when a pressing force of 1 000 kPa or less is applied thereto.

[11] The kit for pretreatment according to [9] or [10], wherein the porous member has a pore having a hole diameter of from 10 μm to 3 000 μm.

[12] The kit for pretreatment according to any one of [9] to [11], wherein the porous member is disposed at a tip portion of a saliva-collecting tool.

[13] The kit for pretreatment according to any one of [9] to [12], wherein the filter has one to four layers.

[14] The kit for pretreatment according to any one of [9] to [13], wherein the device configured to concentrate the antigen or the antibody is an affinity chromatography device of the antigen or the antibody.

[15] The kit for pretreatment according to any one of [9] to [14], wherein a target antigen or a target antibody is SARS-COV-2 or a SARS-COV-2 antibody.

[16] A kit for an immunochromatographic test of an antigen or an antibody in saliva, the kit comprising: the kit for pretreatment according to any one of [9] to [15]; and an immunochromatographic antigen or antibody test kit.

The present invention facilitates to remove only the viscous component in the step (1), to quickly filter the target antigen or the target antibody in the step (2), to sufficiently increase the concentration of the target antigen or the target antibody in the specimen by the step (3), and hence to dramatically improve the sensitivity of detection by an immunochromatographic method. Accordingly, the present invention is particularly advantageous in an immunochromatographic test of SARS-COV-2 or a SARS-COV-2 antibody in saliva.

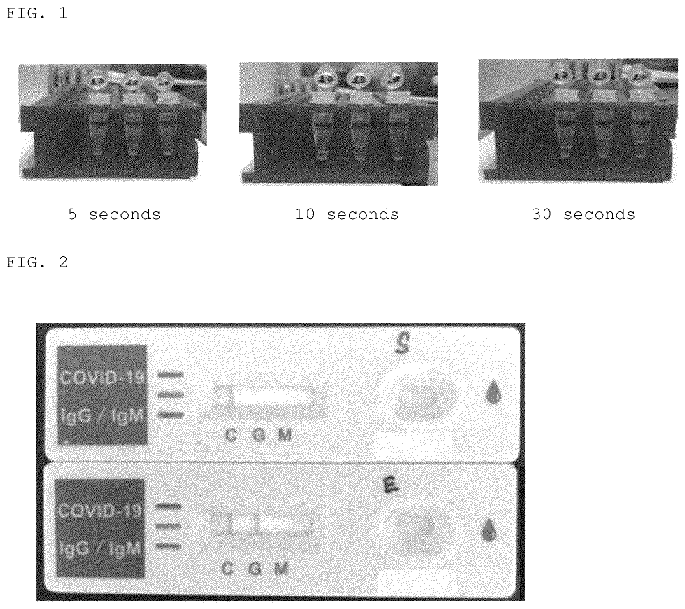

FIG. 1 is a set of views showing results of step (1) and step (2) according to the present invention. The left shows saliva filtered with one filter. The center shows the saliva filtered with two filters. The right (reference) shows a product obtained by adding 10% hexadimethrine bromide to the saliva filtered with one filter at a ratio of 1:1 and mixing the contents for 3 minutes (saliva diluted twofold with hexadimethrine bromide having a final concentration of 5%).

FIG. 2 is a view showing results of step (1), step (2), and step (3) according to the present invention. The upper one shows the result of a filtrate (nonconcentrated specimen), and the lower one shows the result of an eluate (concentrated specimen).

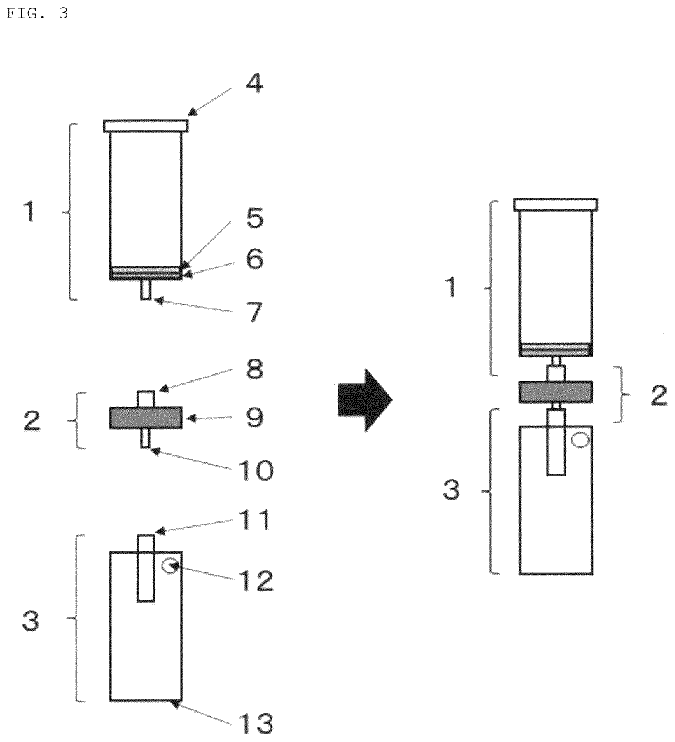

FIG. 3 is a view showing a syringe-type saliva neutralizing antibody concentration recovery instrument. The left side of FIG. 3 shows a state in which the instrument is separated into a filtration unit 1, a saliva neutralizing antibody concentration recovery unit 2, and a saliva filtrate recovery unit 3, and the right side of FIG. 3 shows a state in which these units are inserted into each other.

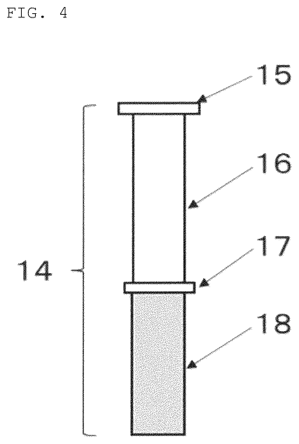

FIG. 4 is a view showing a plunger-type saliva-collecting instrument 14. The instrument has a pressing portion 15, a syringe rod 16, a gasket 17, and a saliva absorber 18.

FIG. 5 is a view showing a state in which the plunger-type saliva-collecting instrument 14 is inserted into the syringe-type saliva neutralizing antibody concentration recovery instrument to perform saliva filtration.

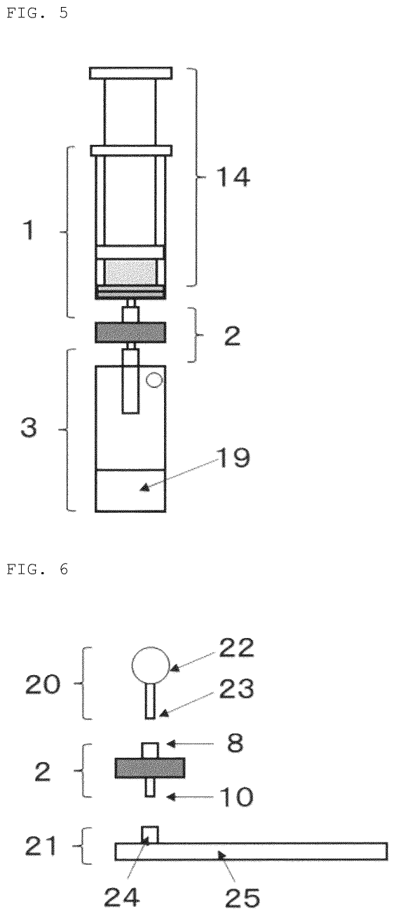

FIG. 6 is a view showing an eluent-containing dropper portion 20 insertable into the saliva neutralizing antibody concentration recovery unit 2, and a saliva antibody immunochromatographic test kit 21 insertable into the saliva neutralizing antibody concentration recovery unit 2.

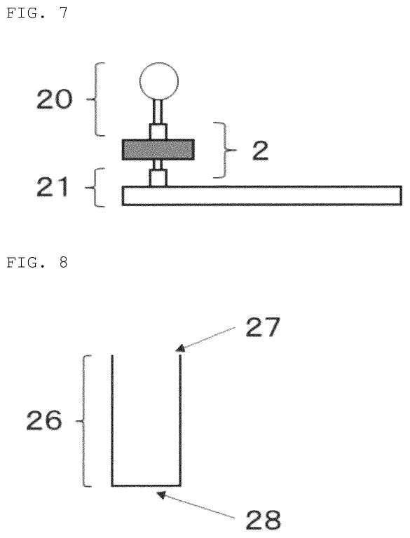

FIG. 7 is a view showing a form in which the eluent-containing dropper portion 20 is inserted into the saliva neutralizing antibody concentration recovery unit 2, and the saliva neutralizing antibody concentration recovery unit 2 is inserted into the saliva antibody immunochromatographic test kit 21.

FIG. 8 is a vertical sectional view of a saliva filtrate recovery unit 26.

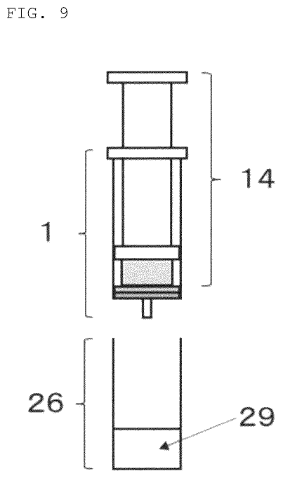

FIG. 9 is a view showing a state in which the plunger-type saliva-collecting instrument 14 is inserted into the filtration unit 1 to perform saliva filtration, followed by the recovery of a saliva filtrate with the saliva filtrate recovery unit 26.

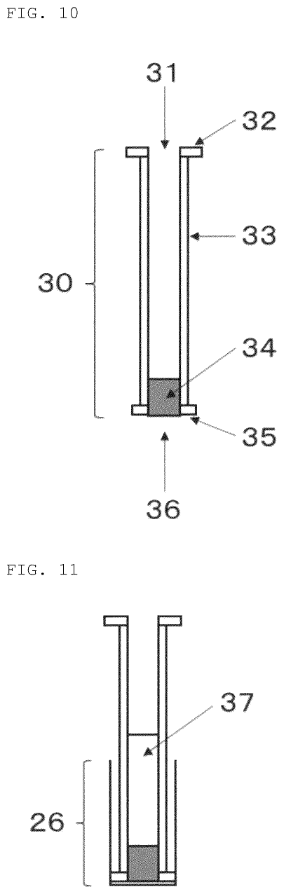

FIG. 10 is a vertical sectional view of a saliva neutralizing antibody concentration recovery unit 30.

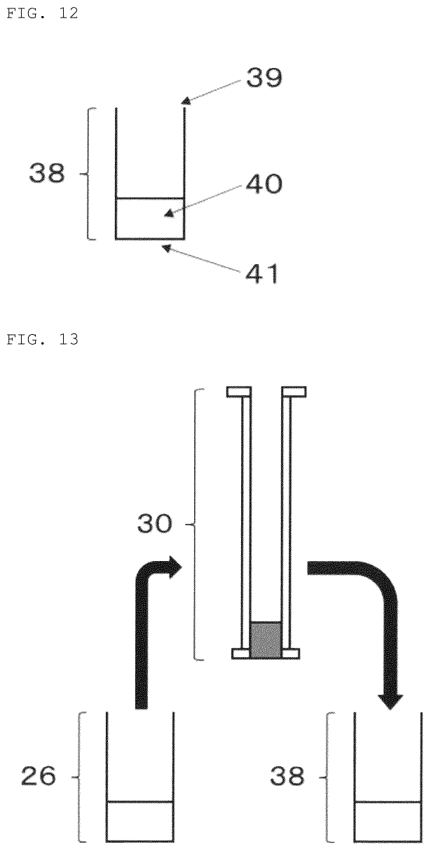

FIG. 11 is a sectional view showing a state in which the saliva neutralizing antibody concentration recovery unit 30 is inserted into the saliva filtrate recovery unit 26 including the saliva filtrate.

FIG. 12 is a vertical sectional view of an eluate container 38.

FIG. 13 is a view showing an operation in which the saliva neutralizing antibody concentration recovery unit 30 is removed from the saliva filtrate recovery unit 26, and is inserted into the eluate container 38.

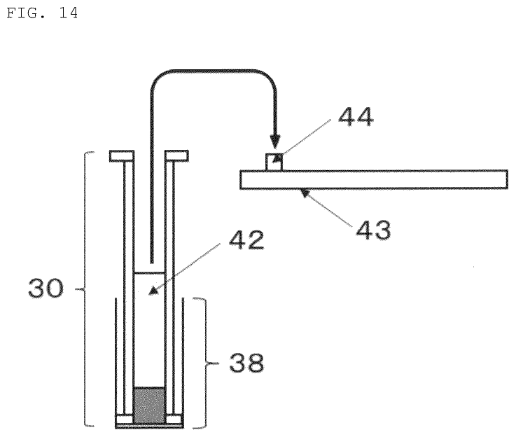

FIG. 14 is a view showing a state in which the saliva neutralizing antibody concentration recovery unit 30 is inserted into the eluate container 38.

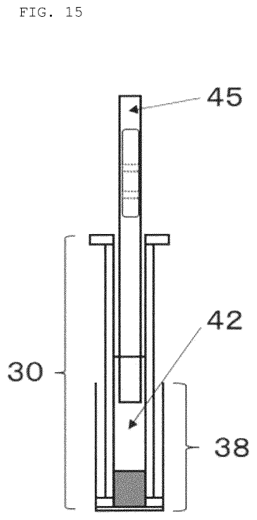

FIG. 15 is a view showing a state in which immunochromatographic test paper 45 is inserted into a neutralizing antibody concentrate 42 obtained in FIG. 14.

### EMBODIMENTS FOR CARRYING OUT THE INVENTION

One embodiment of the present invention is directed to a method for pretreatment of a specimen for an immunochromatographic test, the method including subjecting saliva to: (1) passing the saliva through a porous member capable of supporting a viscous component in the saliva to remove the viscous component; (2) passing the liquid that has passed through the porous member through a filter having a hole diameter of from 0.1 μm to 10 μm; and (3) concentrating an antigen or an antibody in the liquid that has passed through the filter.

The target of the method for pretreatment of the present invention is saliva. The saliva includes: secretions secreted from various salivary glands, such as a parotid gland, a submandibular gland, and a sublingual gland, into an oral cavity; and a gingival crevicular fluid.

Collection of saliva may be performed by the subject on one's own, or may be performed by another person than the subject. At the time of collection of saliva, the saliva is preferably collected after the massage of a gingiva because the gingival crevicular fluid contains a large amount of an antibody. Specifically, the saliva is preferably collected after the secretion of the saliva has been promoted by: sliding a finger from side to side while pressing the finger against upper and lower gingivae over the skin under a state in which the mouth of the subject is closed; and stimulating the inside of his or her oral cavity with his or her tongue.

While the subject may directly discharge the saliva from the oral cavity, the saliva is preferably collected with a saliva-collecting tool.

A dropper or a saliva-collecting tool having, at its tip portion, a porous member capable of supporting a viscous component in the saliva is preferably used as the saliva-collecting tool, and the saliva-collecting tool having, at its tip portion, the porous member capable of supporting the viscous component in the saliva is more preferably used.

The target to be detected by the method for testing of the present invention is an antigen or an antibody in saliva. Examples of the antigen or the antibody may include: various viruses or antiviral antibodies; bacteria or antibacterial antibodies; fungi or antifungal antibodies; and an exogenous antigen or an autoantigen in an immune reaction, or an antibody thereof. Examples of the viruses out of those antigens or antibodies may include an influenza virus, SARS-COV-2, a herpes simplex virus, a rubella virus, a chickenpox virus, a mumps virus, and a cytomegalovirus.

Examples of the bacteria may include Mycoplasma salivarius, Streptococcus mutans, and Treponema pallidum. Further, examples of the fungi may include the genus Candida and the genus Geotrichum.

The target to be detected by the method for testing of the present invention is preferably a SARS-COV-2 or an anti-SARS-CoV-2 antibody, more preferably an anti-SARS-COV-2 antibody.

The collected saliva is subjected to (1) a step of passing the saliva through the porous member capable of supporting the viscous component in the saliva to remove the viscous component (step (1)). The step (1) is important because the viscous component typified by a mucin is present in the saliva, and hence when an attempt is made to filter the saliva with a typical filter, the saliva cannot be filtered.

The porous member capable of supporting the viscous component in the saliva preferably has a void configured to pass a low-viscosity component therethrough without passing a mucin in the saliva therethrough when a pressing force of 1 000 kPa or less is applied thereto. The term “pressing force” as used herein refers to a pressure to be applied when the porous member in a syringe is pressed from above. A porous member having a void configured to pass the low-viscosity component (a liquid including an antigen or an antibody) therethrough without passing the mucin in the saliva therethrough when a pressing force of 500 kPa or less is applied thereto with a plunger or the like is more preferred.

Specifically, a porous member having a pore having a hole diameter of from 10 μm to 3 000 μm is preferred, and a porous member having a pore having a hole diameter of from 10 μm to 1 500 μm is more preferred. Examples of such porous member may include a chemical fiber, cotton, a sponge, and filter paper. Examples of the sponge may include polyurethane-based, polyvinyl alcohol-based, and fluorine-based sponges, and a sponge of a polypropylene-polyethylene polymer.

The porous member is preferably disposed in a saliva-collecting tool (see FIG. 4).

The saliva-collecting tool shown in FIG. 4 is an example of a plunger-type saliva-collecting tool that not only has the porous member but also is intended to apply a pressing force.

The operation of the step (1) is preferably as follows: the saliva is collected with the saliva-collecting tool having, at its tip portion, the porous member capable of supporting the viscous component in the saliva (see FIG. 4); and the viscous component is removed by applying a pressing force with a plunger or the like to pass the saliva through the porous member capable of supporting the viscous component in the saliva (see FIG. 5).

Next step is to perform passing the liquid that has passed through the porous member through the filter having a hole diameter of from 0.1 μm to 10 μm (step (2)). In this step, a liquid containing an antigen, an antibody, and a low-molecular weight component is separated from the liquid that has passed through the porous member.

An example of the filter to be used is a filter for filtering a typical solution or suspension, and examples thereof include a cellulose acetate filter, a polypropylene filter, a nylon filter, and a polytetrafluoroethylene filter. The filter may have one layer, or may have two to four layers each having different hole diameters.

The step (2) is also preferably performed while a pressing force is applied with, e.g., a plunger as shown in FIG. 5.

Next step is to perform concentrating the antigen or the antibody in the liquid that has passed through the filter (step (3)). The concentration of the antigen or the antibody in the liquid that has passed through the filter improves the sensitivity of detection by an immunochromatographic method.

A concentration device, such as an antigen purification kit or an antibody purification kit, is used in the concentration step. Specifically, antigen or antibody affinity chromatography is used. For the antibody, sepharose using a protein bonded to the antibody, such as protein L sepharose or protein G sepharose, is used. For the antigen, sepharose in which protein L, protein G, or the like bonded to the antibody and any other bond are crosslinked with each other is used.

When the affinity chromatography is adopted, the antigen or the antibody is bonded to the concentration device, and hence the antigen or the antibody bonded to the device is eluted and dropped into an immunochromatographic test kit (see FIG. 5 to FIG. 7).

Another embodiment of the present invention is directed to a kit for pretreatment of a specimen for an immunochromatographic test, the kit including: (a) a porous member capable of supporting a viscous component in saliva; (b) a filter having a hole diameter of from 0.1 μm to 10 μm; and (c) a device configured to concentrate an antigen or an antibody.

The pretreatment is preferably performed with the pretreatment kit.

Those described in the step (1), the step (2), and the step (3) may be used as (a) the porous member capable of supporting the viscous component in the saliva, (b) the filter having a hole diameter of from 0.1 μm to 10 μm, and (c) the device configured to concentrate the antigen or the antibody.

The kit for pretreatment of a specimen for an immunochromatographic test of the present invention is described with reference to the drawings.

FIG. 4 shows a plunger-type saliva-collecting instrument 14. The instrument has a pressing portion 15, a syringe rod 16, a gasket 17, and a saliva absorber 18.

FIG. 3 shows a syringe-type saliva neutralizing antibody concentration recovery instrument. The left side of FIG. 3 shows a state in which the instrument is separated into a filtration unit 1, a saliva neutralizing antibody concentration recovery unit 2, and a saliva filtrate recovery unit 3, and the right side of FIG. 3 shows a state in which these units are inserted into each other.

The filtration unit 1 has an upper opening 4 and a lower opening 7, and has a filtration filter 5 and a filtration filter 6. The number of the filtration filters may be one or two or more. With regard to the filter hole diameters of the filtration filter 5 and the filtration filter 6, the hole diameter of the lower filter is preferably smaller than that of the upper filter.

The saliva neutralizing antibody concentration recovery unit 2 has an upper opening 8 and a lower opening 10, and has a neutralizing antibody concentration column 9.

The saliva filtrate recovery unit 3 has an upper opening 11 and a lower closing portion 13, and preferably has an air hole 12.

FIG. 5 shows a view for illustrating a state in which the plunger-type saliva-collecting instrument 14 is inserted into the syringe-type saliva neutralizing antibody concentration recovery instrument 1 to perform saliva filtration. Reference number 19 represents a saliva filtrate.

FIG. 6 shows an eluent-containing dropper portion 20 insertable into the saliva neutralizing antibody concentration recovery unit 2, and a saliva antibody immunochromatographic test kit 21 insertable into the saliva neutralizing antibody concentration recovery unit 2. Reference number 22 represents a dropper sphere portion including an eluent, reference number 23 represents a lower opening from which the eluent is discharged, reference number 24 represents a specimen addition portion of a neutralizing antibody eluted from the saliva neutralizing antibody concentration recovery unit 2, and reference number 25 represents a saliva antibody immunochromatographic test kit. The lower opening 23 has such a structure as to be insertable into the upper opening 8 of the saliva neutralizing antibody concentration recovery unit 2, and the lower opening 10 of the saliva neutralizing antibody concentration recovery unit 2 has such a structure as to be insertable into the specimen addition portion 24.

FIG. 7 shows a view for illustrating a form in which the eluent-containing dropper portion 20 is inserted into the saliva neutralizing antibody concentration recovery unit 2, and the saliva neutralizing antibody concentration recovery unit 2 is inserted into the saliva antibody immunochromatographic test kit 21. When the eluent in the eluent-containing dropper portion 20 is poured into the saliva neutralizing antibody concentration recovery unit 2, the neutralizing antibody in the saliva neutralizing antibody concentration recovery unit 2 is eluted, and the eluate is added to the specimen addition portion 24.

An instrument that concentrates a saliva neutralizing antibody to perform a test in a manner different from that of the instrument illustrated in FIG. 3 to FIG. 7 is illustrated in FIG. 8 to FIG. 15.

FIG. 8 shows a vertical sectional view of a saliva filtrate recovery unit 26. The unit has an upper opening 27 and a lower closing portion 28.

FIG. 9 shows a state in which the plunger-type saliva-collecting instrument 14 is inserted into the filtration unit 1 to perform saliva filtration, followed by the recovery of a saliva filtrate with the saliva filtrate recovery unit 26. Reference numeral 29 represents a saliva filtrate.

FIG. 10 shows a vertical sectional view of a saliva neutralizing antibody concentration recovery unit 30. The saliva neutralizing antibody concentration recovery unit 30 has an upper opening 31 and a lower opening 36 in a hollow tube 33, may have a pressing portion 32 in the upper opening 31, and has a neutralizing antibody concentration column 34 and a gasket 35 in the lower opening 36.

FIG. 11 shows a sectional view for illustrating a state in which the saliva neutralizing antibody concentration recovery unit 30 is inserted into the saliva filtrate recovery unit 26 including the saliva filtrate. Reference number 37 represents the saliva filtrate after r its passage through the saliva neutralizing antibody concentration recovery unit 30.

FIG. 12 shows a vertical sectional view of an eluate container 38. The container has an upper opening 39 and a lower closing portion 41, and reference number 40 represents an eluate.

FIG. 13 shows a view for illustrating an operation in which the saliva neutralizing antibody concentration recovery unit 30 is removed from the saliva filtrate recovery unit 26, and is inserted into the eluate container 38.

FIG. 14 shows a view for illustrating a state in which the saliva neutralizing antibody concentration recovery unit 30 is inserted into the eluate container 38. Reference number 42 represents a neutralizing antibody concentrate eluted from the neutralizing antibody concentration column 34 of the saliva neutralizing antibody concentration recovery unit 30. The resultant neutralizing antibody concentrate 42 may be added as it is as a specimen to the opening 44 of a saliva antibody immunochromatographic test kit 43.

FIG. 15 shows a view for illustrating a state in which immunochromatographic test paper 5 is inserted into the neutralizing antibody concentrate 42 obtained in FIG. 14.

Still another embodiment of the present invention is directed to a method for an immunochromatographic test of an antigen or an antibody in saliva, including subjecting a specimen obtained by the above-mentioned pretreatment step to an immunochromatographic antigen or antibody test kit.

Herein, an immunochromatographic test kit with colored latex particles for detecting a target antibody is simply described. A typical immunochromatographic test kit includes: a sample addition portion; a target antigen-labeling colored particle-holding portion; and an antibody-detecting portion having fixed thereto a target antigen. First, the specimen obtained by the pretreatment is dropped into the specimen addition portion, and the specimen is developed toward the target antigen-labeling colored particle-holding portion. When the target antibody of interest is mixed into the specimen, the target antibody and the target antigen-labeling colored latex particles react with each other, and a composite of the antibody and the particles is captured by the antibody-detecting portion having fixed thereto the target antigen. Thus, a colored band appears. The amount of the target antibody in the specimen can be roughly grasped by, for example, the color tone of the band that has appeared.

Still another embodiment of the present invention is directed to a kit for an immunochromatographic test of an antigen or an antibody in saliva, the kit including: (a) the porous member capable of supporting a viscous component in the saliva; (b) the filter having a hole diameter of from 0.1 μm to 10 μm; (c) the device configured to concentrate an antigen or an antibody; and (d) an immunochromatographic antigen or antibody test kit.

Those described in the step (1), the step (2), and the step (3) may be used as (a) the porous member capable of supporting the viscous component in the saliva, (b) the filter having a hole diameter of from 0.1 μm to 10 μm, and (c) the device configured to concentrate the antigen or the antibody. In addition, the typical immunochromatographic test kit may be used as (d) the immunochromatographic antigen or antibody test kit.

In the present invention, the method for pretreatment of a specimen for an immunochromatographic test and the method for an immunochromatographic test of an antigen or an antibody in immunochromatographic saliva thereof are each particularly useful as a method for a test of an anti-SARS-COV-2 antibody for which a simple and highly sensitive detection method has heretofore been absent, the method including use of saliva.

### EXAMPLES

Next, the present invention is further described by way of Examples. However, the present invention is not limited to these Examples, and various modifications are possible within a range not departing from the technical gist of the present invention.

Abbreviations used in Examples below represent the following meanings.
Syringe: Terumo Disposable Syringe 5 mL
Filter: Advantec Syringe Filter (0.80 μm)
Nanosep: Nanosep Centrifugal Filtration Device (0.45 μm)
Saliva absorber (porous member): A swab taken out of Salivette (saliva-collecting kit, SARSTEDT K.K.)
Antibody purification kit: A spin column-based IgG antibody purification kit (Cosmo Bio Company, Limited) APK-10G
Antibody measurement kit: KBM COVID-19 IgG/IgM (whole blood·plasma·serum, Kohjin Bio Co., Ltd.)

### Comparative Example 1

The secretion of saliva was promoted by: performing gingival massage for 2 minutes by sliding a finger from side to side while pressing the finger against upper and lower gingivae over the skin with the mouth of a subject being closed; and at the same time, stimulating the inside of his or her oral cavity such as gingivae with his or her tongue. 3 mL of the saliva was collected in a paper cup. 2 mL of the collected saliva was loaded into a syringe mounted with one filter, and a pressure was applied thereto with a plunger.

As a result of the test, the saliva did not pass through the filter with the pressure applied to the plunger.

### Example 1

In the same manner as in Comparative Example 1, 3 mL of saliva was collected in a paper cup, and a swab (saliva absorber) that had been softened was caused to absorb the saliva. The swab that had absorbed the saliva was loaded into a syringe coupled with one or two filters, and a pressure was applied with a plunger to filter the saliva. The filtrate was recovered, and 500 μL of the filtrate was added to Nanosep, followed by centrifugation with a desktop small centrifuge at 4 000 rpm for 5, 10, or 30 seconds. The saliva was evaluated for its viscosity with its permeation amount through the Nanosep as an indicator. The results are shown in FIG. 1.

As a result of the test, the saliva filtered with the filter twice showed permeability higher than that of the saliva filtered therewith once.

### Example 2

As with Example 1, a swab (saliva absorber) was caused to absorb saliva. The swab that had absorbed the saliva was loaded into a syringe coupled with two filters, and a pressure was applied with a plunger to filter the saliva. The filtrate was recovered, and 500 μL of the filtrate was passed through the column of an antibody purification kit. Subsequently, the filtrate was eluted with 50 μL of a buffer included in the kit (tenfold concentration: theoretical value), and 20 μL of the eluate (concentrated specimen) was dropped into an antibody measurement kit for blood, followed by comparison to 20 μL of the filtrate (nonconcentrated specimen). The results are shown in FIG. 2.

As a result of the test, no IgG antibody was detected in the nonconcentrated specimen, but an IgG antibody was detected in the concentrated specimen.

### Example 3

After the same gingival massage as that of Comparative Example 1 has been performed, a plunger-type saliva-collecting instrument (FIG. 4) having a soft saliva absorber attached to its tip is included in an oral cavity to collect saliva. The saliva-collecting instrument that has absorbed the saliva is inserted into a syringe-type filtration concentration instrument having incorporated thereinto a filtration filter and an antibody concentration column, and a pressure is applied with a plunger to filter the saliva through a filter (FIG. 5). At the same time, an antibody in the filtrate is concentrated with the antibody concentration column. The antibody concentration column is taken out of the filtration concentration instrument and mounted on an immunochromatographic plate, and a tube containing an eluent (e.g., a citrate buffer) is attached to the upper portion of the antibody concentration column (FIG. 7). Subsequently, a pressure is applied to the periphery of the tube to elute the antibody, and several drops of the eluate are dropped into the specimen addition portion of an immunochromatographic test kit.

### REFERENCE SYMBOLS LIST

1: filtration unit
2: saliva neutralizing antibody concentration recovery unit
3: saliva filtrate recovery unit
4: upper opening of filtration unit
5: filtration filter
6: filtration filter
7: lower opening of filtration unit
8: upper opening of saliva neutralizing antibody concentration recovery unit
9: neutralizing antibody concentration column
10: lower opening of saliva neutralizing antibody concentration recovery unit
11: upper opening of saliva filtrate recovery unit
12: air hole
13: lower opening of saliva filtrate recovery unit
14: plunger-type saliva-collecting instrument
15: pressing portion
16: syringe rod
17: gasket
18: saliva absorber
19: saliva filtrate
20: eluent-containing dropper portion
21: saliva antibody immunochromatographic test kit
22: dropper sphere portion including eluent
23: lower opening from which eluent is discharged
24: specimen addition portion of neutralizing antibody
25: saliva antibody immunochromatographic test kit
26: saliva filtrate recovery unit
27: upper opening of saliva filtrate recovery unit
28: lower opening of saliva filtrate recovery unit
29: saliva filtrate
30: saliva neutralizing antibody concentration recovery unit
31: upper opening of saliva neutralizing antibody concentration recovery unit
32: pressing portion
33: hollow tube
34: neutralizing antibody concentration column
35: gasket
36: lower opening of saliva neutralizing antibody concentration recovery unit
37: saliva filtrate
38: eluate container
39: upper opening
40: eluate
41: lower opening
42: neutralizing antibody concentrate
43: saliva antibody immunochromatographic test kit
44: opening of kit
45: immunochromatographic test paper

1. A method for pretreatment of a specimen for an immunochromatographic test, comprising:
passing the saliva through a porous member capable of supporting a viscous component in the saliva to remove the viscous component to obtain a liquid;
passing the liquid through a filter having a hole diameter of 0.1 to 10 μm; and
concentrating a target antigen or a target antibody in the liquid.

2. The method of claim 1, wherein the porous member has a void configured to pass a low-viscosity component but not to pass a mucin in the saliva therethrough when a pressing force of 1000 kPa or less is applied thereto.

3. The method of claim 1, wherein the porous member has a pore having a hole diameter of 10 to 3000 μm.

4. The method of claim 1, wherein the porous member is disposed at a tip portion of a saliva-collecting tool.

5. The method of claim 1, wherein the filter has one to four layers.

6. The method of claim 1, wherein the concentrating comprises the target antigen or the target antibody undergoing affinity chromatography.

7. The method of claim 1, wherein the target antigen or the target antibody is an antigen of SARS-COV-2 or a SARS-COV-2 antibody.

8. A method for an immunochromatographic test of an antigen or an antibody in saliva, comprising:
subjecting a specimen obtained by the method of claim 1 to an immunochromatographic antigen or antibody test kit.

9. A kit for pretreatment of a specimen for an immunochromatographic test, comprising:
a porous member capable of supporting a viscous component in saliva;
a filter having a hole diameter of 0.1 to 10 μm; and
a device configured to concentrate a target antigen or a target antibody.

10. The kit of claim 9, wherein the porous member has a void configured to pass a low-viscosity component but not to pass a mucin in the saliva therethrough when a pressing force of 1000 kPa or less is applied thereto.

11. The kit for pretreatment according to of claim 9, wherein the porous member has a pore having a hole diameter of 10 to 3 000 μm.

12. The kit of claim 9, wherein the porous member is disposed at a tip portion of a saliva-collecting tool.

13. The kit of claim 9, wherein the filter has one to four layers.

14. The kit of claim 9, wherein the device configured to concentrate the target antigen or the target antibody is an affinity chromatography device of the antigen or the antibody.

15. The pretreatment kit of claim 9, wherein a target antigen or a target antibody is an antigen of SARS-COV-2 or a SARS-COV-2 antibody.

16. A kit for an immunochromatographic test of an antigen or an antibody in saliva, comprising:
the kit of claim 9; and
an immunochromatographic antigen or antibody test kit.
